(19) United States
(12) Patent Application Publication
(10) Pub. No.: US 2023/0277385 A1
(43) Pub. Date: Sep. 7, 2023

(54) CONFORMABLE MEDICAL DRESSING

(71) Applicant: 3M INNOVATIVE PROPERTIES COMPANY, St. Paul, MN (US)

(72) Inventors: Guido Hitschmann, Neuss (DE); Benjamin Vollmann, Recklinghausen (DE); Richard L. Jacobson, Stillwater, MN (US); Daniel Dawidowski, Unna (DE)

(21) Appl. No.: 18/015,902

(22) PCT Filed: Jul. 14, 2021
(86) PCT No.: PCT/IB2021/056350

§ 371 Date: Jan. 12, 2023

Related U.S. Application Data
(60) Provisional application No. 63/052,556, filed on Jul. 16, 2020.

(51) Int. Cl. A61F 13/02

(52) U.S. Cl. CPC A61F 13/0226; A61F 2013/00412

(57) ABSTRACT

The disclosed medical dressing includes a dressing body with an overlying carrier system that is releasably secured to the dressing body. The carrier has a perimeter carrier portion overlying the perimeter of the dressing body and a central carrier portion overlying some, but not all, of a central area of the dressing body. This carrier construction allows for better handling of the medical dressing during application by giving both stability in some areas and stretching and flexibly in areas without the carrier.

### TECHNICAL FIELD

The present disclosure relates to a medical dressing with an overlying removable carrier system.

### BACKGROUND

Thin transparent film dressings are widely used on skin because they provide protection and act as a barrier to contaminating liquids and bacteria. For example, transparent film dressings are available under trade names such as TEGADERM™ (3M Company, St. Paul, Minn.). The polymeric films used in those dressings and drapes are conformable, i.e., the films are extremely thin, flexible and supple. They are typically supplied with a releasable protective liner covering the adhesive coated surface of the film. To prevent the adhesive coated film from wrinkling during application, various delivery systems, such as a removable carrier, have been proposed, such as those disclosed in U.S. Pat. No. 6,685,682.

Such transparent film dressings can be designed to receive and help secure devices such as tubing, ports and catheters, and the dressing may provide additional securement devices to stabilize such devices. Given the multi-component structure of such dressings, application can involve multiple steps and mistakes can lead to wasted materials and patient discomfort.

### SUMMARY

The disclosed medical dressing includes a dressing body with an overlying carrier system that is releasably secured to the dressing body. The carrier has a perimeter carrier portion overlying the perimeter of the dressing body and a central carrier portion overlying some, but not all, of a central area of the dressing body. This carrier construction allows for better handling of the medical dressing during application by giving both stability in some areas and stretching and flexibly in areas without the carrier.

In one embodiment, the medical dressing has a dressing body, a pressure sensitive adhesive on a bottom major surface of the dressing body, and a carrier releasably secured to a top major surface of the dressing body. The dressing body typically is a thin film construction that advantageously is highly conformable to the underlying surface. In addition, a thin film construction can be moisture-vapor permeable to prevent skin maceration. However, such thin film constructions are difficult to handle Therefore, the carrier is included to support the dressing body during application. The carrier a peripheral carrier portion and a central carrier portion. The peripheral carrier portion is adjacent to the perimeter of the dressing body to keep the dressing body planar and wrinkle-free for application to a surface. The central carrier portion is adjacent to the first dressing area. Similarly, this keeps the underlying dressing body 150 planar and wrinkle-free until the central carrier portion is removed. The second dressing area does not contain an overlying carrier. The dressing body remains exposed at the top major surface at the second dressing area. At least partially surrounding the second dressing area is at least one or both of the peripheral carrier portion and central portion. Keeping the second dressing area free from the carrier gives flexibility of the dressing body before and during application of the medical dressing 100 to an underlying surface.

In one embodiment, the second dressing area is at least partially surrounded by the peripheral carrier portion and the central carrier portion. In one embodiment, at the second dressing area, the dressing body forms an outermost surface of the medical dressing. The first dressing area, typically does not contain reinforcing layer, and may extend beyond the central carrier portion.

In one embodiment, the dressing body further comprises a slot, which is a recess of the dressing perimeter at the second dressing area.

In one embodiment, the dressing body is a single or multilayer film. In one embodiment, the multilayer film of the dressing body at the first dressing area comprises a moisture-vapor permeable film and at the second dressing area comprises a reinforcing layer and a moisture vapor permeable and water impermeable film.

In one embodiment, at least a portion of the central carrier portion is directly adjacent to the peripheral carrier portion. In one embodiment, the central carrier portion is connected with the peripheral carrier portion by an area of weakness. In one embodiment, the area of weakness is a partial thickness cut or a perforation. In one embodiment, the central carrier portion further comprises a central carrier tab extending into the second dressing area. In one embodiment, the peripheral carrier portion further comprises a peripheral carrier tab extending into the second dressing area. In one embodiment, adhesive fixation strips are at the top major surface secured to the central carrier portion and the peripheral carrier portion. In one embodiment, the peripheral carrier portion further comprises an area of weakness (e.g. S shaped cutting line or perforation) with or without tab to aid in removal of the peripheral carrier portion.

In one embodiment, a method applying the medial dressing includes the steps of placing the pressure sensitive adhesive of the dressing body onto a surface, removing the central carrier portion, and removing the peripheral carrier portion.

In one embodiment, a method applying the medial dressing includes the steps of removing the central carrier portion, placing the pressure sensitive adhesive of the dressing body onto a surface, and removing the peripheral carrier portion.

In one embodiment, the method of applying the dressing comprising pulling the central carrier tab to remove the carrier central portion. In one embodiment, the method of applying the dressing further comprises pulling the peripheral carrier tab to remove the peripheral carrier portion.

As used herein, “a” “an,” “the,” “at least one” and “one or more” are used interchangeably. The term “and/or” (if used) means one or all of the identified elements or a combination of any two or more of the identified elements. The term “comprises”, and variations thereof do not have a limiting meaning where those terms appear in the description and claims. Also herein, the recitations of numerical ranges by endpoints include all numbers subsumed within that range (e.g. 1 to 5 includes 1, 1.5, 2, 2.75, 3, 3.80, 4, 5, etc.). Any patents referred to herein are hereby incorporated by reference in their entirety.

### BRIEF DESCRIPTION OF DRAWINGS

FIG. 1 is a perspective view of a first embodiment of the medical dressing;

FIG. 2 is a side sectional view of the medical dressing of FIG. 1 through line 2-2;

FIG. 3 is a perspective view of a dressing body in a further embodiment of the disclosure; and

FIG. 4 is a top view of second embodiment of the medical dressing having two tabs for removal of the carrier central portion.

While the above-identified drawings and figures set forth embodiments of the invention, other embodiments are also contemplated, as noted in the discussion. In all cases, this disclosure presents the invention by way of representation and not limitation. Numerous other modifications and embodiments can be devised by those skilled in the art, which fall within the scope and spirit of this invention. The figures may not be drawn to scale.

### DETAILED DESCRIPTION

The disclosed medical dressing includes a dressing body with an overlying carrier system that is releasably secured to the dressing body. The carrier has a perimeter carrier portion overlying the perimeter of the dressing body and a central carrier portion overlying some, but not all, of a central area of the dressing body. This carrier construction allows for better handling of the medical dressing during application by giving both stability in some areas and stretching and flexibly in areas without the carrier.

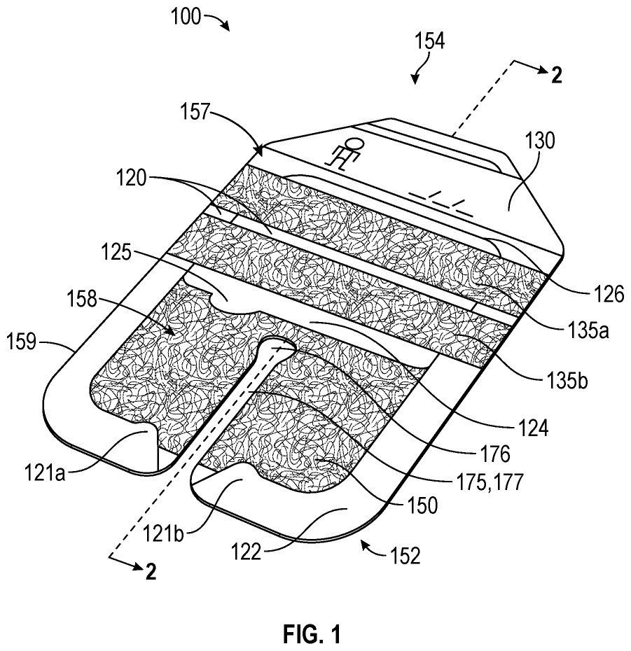

Specific embodiments will be described in more detail. As shown in FIG. 1, the medical dressing 100 comprises a dressing body 150 with a slot 175 for receiving a device. In the embodiment shown in FIG. 1, the slot 175 is shown having a larger head portion 176, and a narrower body portion 177, and various configurations can be contemplated depending on the type of device the dressing is designed to secure. The dressing body 150 has a bottom major surface 152 and an opposite top major surface 154, wherein at least a portion of the bottom major surface 152 is coated with a pressure sensitive adhesive 153 to secure the dressing body 150 to an underlying surface, such as skin. The bottom major surface 152 of the dressing 100 may contain a reinforcement material 140 adhered to at least a portion of the bottom major surface 152 of the dressing body 150. The dressing body 150 has a perimeter 159 that defines a first dressing area 157 and second dressing area 158.

At least a portion of the top major surface 154 of the dressing body 150 is removably attached to a carrier 120 to help maintain the dressing body 150 in a flat, unwrinkled and unstretched position as it is applied to the patient. Atop a portion of the carrier 120 is a label 130, wherein the label 130 contains printing on its top surface, e.g., to provide prompts for entering patient information. Also, atop a portion of the carrier 120 are two fixation strips, 135a and 135b, to help secure a device such as tubing, ports and catheters that are received in the slot 175 of the dressing body 150.

The carrier 120 comprises a peripheral carrier portion 122 and a central carrier portion 124. Typically, the peripheral portion 122 is connected to the central portion 124 by a line of weakness which is either a partial cut or a set of perforations 126. The line of weakness helps hold the peripheral carrier portion 122 to the central carrier portion 124 until removal is desired.

In the embodiment shown, the central carrier portion 124 of the carrier 120 does not cover the entire area of the dressing body 150, a second area 158 is a free portion without the overlying carrier. This free portion has adjacent carrier 120, and in this embodiment carrier 122, 124 that nearly completely surrounds the second area 158. This surrounding carrier 120 provides structural support to the dressing body 150 to prevent wrinkling. In one embodiment, the surrounding carrier 120, comprising peripheral carrier portion 122, and central carrier portion 124, might represent more than 40% and less than 95% of the dressing area 150. This configuration enables handling of the dressing 100 in a wrinkle free and unstretched position while also facilitating easy removal if the dressing body 150 is applied to the patient before the carrier 120 is removed.

Optional carrier tab 125 extends from the central carrier portion 124 into the second area 158 to facilitate removal of the central carrier portion 124 of the carrier 120. Optionally, peripheral carrier tabs 121a and 121b extend from the peripheral carrier portion 122 into the central carrier portion 124 to facilitate removal of the peripheral portion 122 of the carrier 120. In the embodiment shown in FIG. 1, central carrier tab 125 is shown on the left side of the central portion 124 of the carrier 120 and is spaced away from the peripheral portion 122 of the carrier 120. Central carrier tab 125 may be placed anywhere along the central carrier portion 124 into the second area 158 and spaced from the peripheral portion 122 or adjacent to it. Additionally, there may be more than one central carrier tab for removing the central carrier portion 124 of the carrier 120.

FIG. 4 shows an alternate design for including two central carrier tabs 425a, 425b on the central portion 424 to facilitate removal of the central carrier portion 424. Peripheral tabs 421a and 421b aid in removal of the peripheral portion 422 of the carrier 420 and have a rounded edge removed from the edge of the dressing body 450 for ease of removal. Other shapes and designs might be used to facilitate removal of the peripheral portion 122 of the carrier 120.

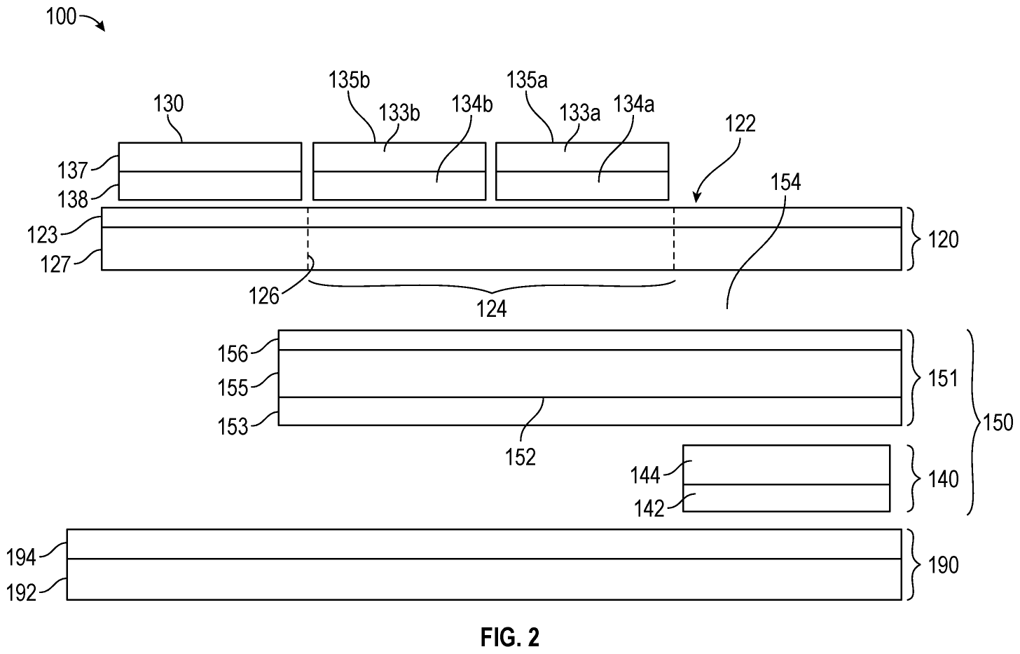

FIG. 2 is a side sectional view of the dressing 100 of FIG. 1 along line 2-2, showing further detail of the dressing 100 components. The dressing body 150 comprises a laminate 151 and an optional reinforcing material 140. In one embodiment, the laminate 151 comprises a thermoplastic polyurethane with optionally additional layers of polymeric layers, fabric, or reinforcing layers. The laminate 151 comprises a film layer 155 having a top major surface 154 and a bottom major surface 152. A pressure sensitive adhesive (PSA) 153 is coated on a portion of the bottom major surface 152 of the film layer 155, and a low-adhesive backing (LAB) 156 is coated on the top major surface 154 of the film layer 155. The LAB backing 156 facilitates the release of the carrier 120, from the top major surface 154 of the laminate 151.

In this embodiment, at least a portion of the bottom major surface 152 of the laminate 151 is adhered via a pressure sensitive adhesive 153 to a reinforcement material 140. In this embodiment, the reinforcing material 140 is at the second area 158. Typically, the reinforcing material 140 is not at the first area 157. At the first area 157, often just the laminate is included to provider better transparency, conformability. In an embodiment, the lower edge of the reinforcement material 140 is inset from perimeter of the laminate 151. In this embodiment, the reinforcement material 140 comprises a nonwoven layer 144 having a bottom surface at least partially coated with a pressure sensitive adhesive 142. The adhesive 142, together with the adhesive 153 on the bottom major surface 152 of the laminate 151 facilitates the adhesion of the dressing body 150 to the patient. The laminate 151 and reinforcing layer 140 are the components that remain on the patient after the dressing 100 is applied. The label 130 and fixation strips 135a, b can also remain on the patient, if the user desires to use them so.

Turning now to the carrier 120, which is used to stabilize the dressing body 150 and keep it wrinkle-free and without stretch during handling and to apply the dressing without touching the adhesive. Therefore, laminate 151 is recessed from carrier 120. The top surface of the carrier 120 is coated with a LAB 123 to facilitate the release of the label 130 and fixation strips 135 a, b. The carrier substrate 127 may be made of a suitable paper or polymer material of sufficient rigidity to provide adequate support. A line of weakness 126 can be between the central carrier portion 124 and the adjacent peripheral portion 122. The line of weakness 126 can be a partial cut through the depth of the carrier substrate 127 allowing for easy separation of the central carrier portion 124 from the peripheral portion 122. In another embodiment, the line of weakness 126 can be a set of perforations cuts through the depth of the carrier substrate 127. Keeping some attachment between the central carrier portion 122 and the peripheral portion 124 hold the integrity of the carrier 120 together until separation is desired.

In an embodiment, two fixation strips 135a and 135b are shown secured to the top major surface 154. The fixation strips 135a, b are an adhesive tape. In this embodiment, the fixation strips 135a, b comprise a soft-cloth material 133 a, b, respectively, with an adhesive 134 a, b respectively, on the bottom surface of each fixation strip 135 a, b. The adhesive layer 134 a, b of the fixation strips 135 a, b are adhered to the carrier 120 with the ends of the strips extending beyond the carrier central portion 124. The fixation strips 135a, b secure to at least a portion of the peripheral carrier portion 122 and the central carrier portion 124. Therefore, upon removal of the central carrier portion 124, an end of the fixation strip 135a, b is free to easily remove it and use it. In this embodiment, the fixation strips 135a, b extend entirely across the central carrier portion 124 on to two opposing sides of the peripheral carrier portion 122.

An optional label 130 which is any suitable label tape material 137 coated on at least a portion of its bottom surface with an adhesive 138. The top surface of the label tape material 137 may contain preprinted information, such as prompts to enter patient information. The top surface of the label tape material 137 may be a material that can be written on with a maker.

In an embodiment, at least a portion of one or more of the adhesive layers 138, 153 and 142 of the label 130, laminate 151, and optional reinforcement soft cloth 140, respectively, are protected by a product liner 190 to protectively cover the adhesive prior to use, but is easily removed prior to use. In this embodiment, the product liner 190 comprises a liner paper 192 having an LAB coating 194 on its top surface major surface which comes into contact with the adhesive layers 138, 153 and 142. The surfaces of adhesive layers 153 and 142 collectively provide the adhesion to secure the dressing body 150 to the patient.

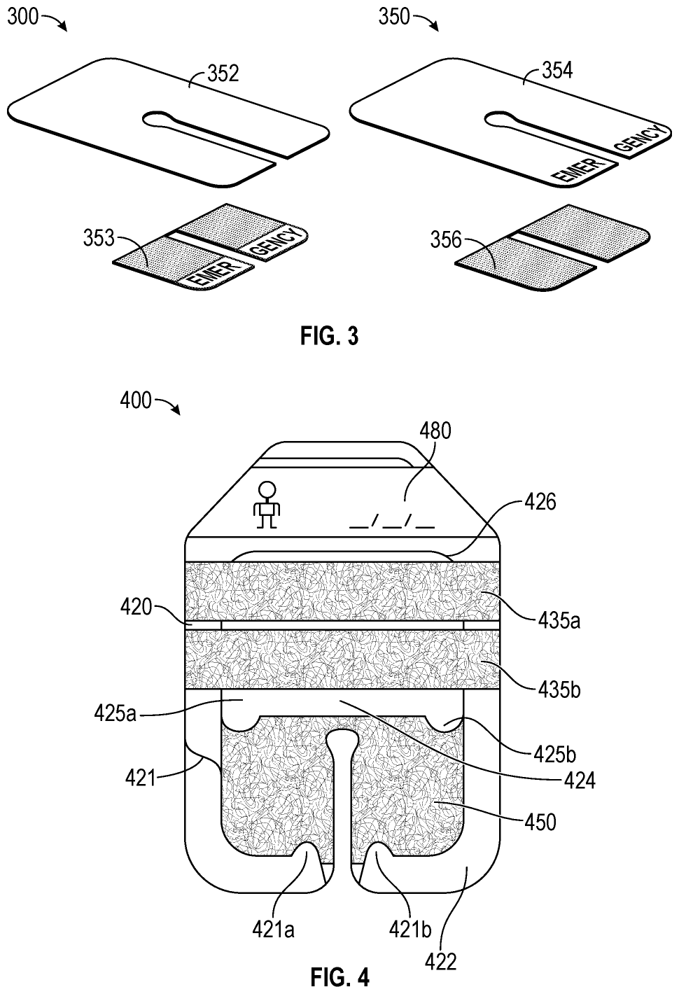

In further embodiments shown in FIG. 3, alternate designs for the laminate and reinforcing layer are shown. Dressing bodies 300 and 350 are shown having a transparent film layers 352 and 354, and reinforcing layers 353 and 356. In these embodiments, a portion of the top surface of the layers 352, 354 or the soft cloth layers 353, 356 contain printing on a portion of their respective surfaces. Such printing can contain various words, symbols, QOR codes, images, etc., which may convey information about the patient. For example, the writing may indicate that the dressing was placed on the patient by emergency personnel. Some jurisdictions or medical guidelines require that such dressings be changed once the patient enters the hospital, thus, such a marking can facilitate compliance with such requirements.

FIG. 4 shows a medical dressing 400 in a further embodiment which is substantially similar to the medical dressing 100. The medical dressing 400 has a carrier 420 with a central portion 424 and peripheral portion 422, wherein the central portion 424 is connected to the peripheral portion 422 by a line of weakness or perforation 426. In this embodiment, different than FIG. 1, the central portion 424 contains two central portion tabs 425a, b, which are adjacent to the peripheral portion 422 of the carrier 420. As can be appreciated, central portion tabs 425 a, b can also be spaced from the peripheral portion 422 of the carrier 420. Peripheral portion tabs 421 a, b facilitate removal of the peripheral portion 422 of the carrier. Similar to FIG. 1, the medical dressing 400 further contains fixation strips 435a, b and label 480.

In this embodiment, at the peripheral carrier portion 422 is an area of weakness 421 partially or entirely through the peripheral carrier portion 422. This area of weakness 421 can be a partial or complete through cut in the peripheral carrier portion 422. In the application, the user could pinch this section with the fingers and can start removal of the peripheral carrier portion 422. An advantage of the renovation portion here is that it is away from the notch, where pinching and pulling of the peripheral carrier portion 422 in this location could move and irritate the catheter.

Application of the medical dressing to a surface will be described with reference to the embodiment described in FIG. 1. One way of applying the medical dressing 100 to a substrate includes securing the medical dressing 100 to a surface. Then, once secured, the user can remove the central carrier portion 124. Removal of the carrier central portion 124 will release the overlying fixation strips 135a, b, if included. Then, the peripheral carrier portion 122 is removed leaving the secured medical dressing 100 on the substrate.

This method of applying the dressing in more detail. Typically, after removing the dressing 100 from its sterile packaging, the user removes the product liner 190 to adhere the exposed adhesive surfaces (153, 142) to the surface, such as a patient's skin, to secure devices such as tubing, ports, and catheters. Application of pressure to the top surface 154 of the dressing body 150 will ensure proper adhesion to the patient. Next, the user removes the carrier central portion 124 with the fixation strips 135a, b by pulling tab 125 to release the central portion 124 from the peripheral portion 122 of the carrier 120. By this, the line of weakness 126 is broken. Then, the user removes the carrier peripheral portion 122 from the top surface 154 of the dressing body 150 using one or both tabs 121a, b found on the lower edge of the slot 175 for receiving a device. The user may then remove one or both of the fixation strips 135a, b from the carrier central portion 124 to further secure catheter hub, lumen or tubes. In this method, the fixation strips 135a, b may be fully or partially placed on top of the dressing body 150, which has been secured to the patient. Alternatively, fixation strips 135a, b may be used to secure device(s) and be placed directly on the skin of the patient Once the dressing body 150 and fixation strips 135a, b are in place and after the carrier portions are removed, application of pressure to the periphery of the dressing body 150 will ensure proper adhesion. Finally, the user removes the label 130 from the carrier peripheral portion 122, enters patient information, and adheres the label 130 to the patient near or on top of the dressing body 150. The use of one or both of the fixation strips 135a, b and label 130 are optional and may or may not be used depending on the needs of the particular application.

In a second method of applying the medical dressing 100 to a substrate, the user may first remove the carrier central portion 124 to release the fixation strips 135a, b and apply them to secure devices such as tubing, ports, and catheters. Then, the medical dressing 100 is applied to the substrate. Because the peripheral carrier portion 122 remains, the medical dressing 100 is still supported so that it doesn't wrinkle and collapse on itself. The peripheral carrier portion 122 is then removed leaving the secured medical dressing 100 on the substrate.

This second method will be described in more detail. Typically, the user first removes the dressing 100 from its sterilized outer packaging (not shown). Next, the user removes carrier central portion 124 using tab 125, which releases fixation strips 135a, b, from the peripheral carrier portion 122. Doing this, the line of weakness 126 is broken. Fixation strips 135a, b can then be removed from the carrier central portion 124 and applied to the patient to secure devices such as tubing, ports, and catheters. The carrier central portion 124 is then discarded. Once the device(s) are secure, the dressing body 150 can be applied to the patient by first removing the product liner 190 and applying the dressing body 150 to the patient over the fixation strips 135a, b and placing slot 175 around the device(s). To avoid touching the adhesive, the carrier 120 extends beyond the laminate 151, opposite side of the slot 175 (opposite side of the slot 175).

Applying pressure to the dressing body 150 will ensure an adequate bond. In some instances, medical dressings are applied to a patient and remain in place for several days. When dressings are worn over time, the edges of the dressing can begin to peel away from the patient possibly resulting in contamination at the site or adhesive failure entirely. Applying pressure to the adhesive results in a stronger bond and lessens the possibility of adhesive failure.

Once the dressing body 150 has been applied to the patient, the carrier peripheral portion 122 is removed from the top surface 154 of the dressing body 150 using one or both tabs 121a, b found next to the lower, inner edge of the slot 175 for receiving a device. After removing the carrier peripheral portion 122, pressure is applied to the periphery of the dressing body 150 in order to ensure proper adhesion. In the embodiment, shown in cross section in FIG. 2, the dressing 100 has the reinforcing material 140 inset from the lower edge of the film laminate 151 to prevent the dressing body 150 from rolling up from the lower edge. The user can remove the label 130 from the carrier peripheral portion 122, enter patient information, and adheres the label 130 to the patient near or on top of the dressing body 150. The use of one or both of the fixation strips 135a, b and label 130 are optional and may or may not be used depending on the needs of the particular application.

The easily accessed central portion 124 of the medical dressing 100 described herein allows the user to employ different methods of application with a single product. The central portion 124 can serve to add stability to the underly dressing and also can deliver the overlying fixation strips, if included. Inclusion of the second area of the dressing that does not have the overlying carrier gives flexibility during application of the medical dressing, especially when applying over and around a medical device such as tubing. The adjacent peripheral carrier portion and central carrier portion provide the peripheral support to keep this second area of the dressing wrinkle free until application to the surface.

Dressing Body and Optional Reinforcing Material

The dressing body is typically formed of thin film materials, which provide resistance against incoming water and contaminants and high moisture vapor permeability to allow moisture vapor from the underlying skin to exit and conformability to surfaces. Typical thicknesses for such films is about 20 μm to 40 μm.

One example of a suitable material is a high moisture vapor permeable film such as described in U.S. Pat. Nos. 3,645,835 and 4,595,001, the disclosures of which are herein incorporated by reference. Issued U.S. Pat. Nos. 3,645,835 and 4,595,001, the disclosures of which are hereby incorporated by reference, describe methods of making such films and methods for testing their permeability. Typically, the film/adhesive composite should transmit moisture vapor at a rate equal to or greater than human skin. Typically, the adhesive coated film transmits moisture vapor at a rate of at least 300 g/m²/24 hrs/37° C./100-10% RH, more preferably at least 700 g/m²/24 hrs/37° C./100-10% RH, and most preferably at least 2000 g/m²/24 hrs/37° C./100-10% RH using the inverted cup method as described in U.S. Pat. No. 4,595,001.

The material for the dressing is preferably conformable to anatomical surfaces. As such, when applied to an anatomical surface, it conforms to the surface even when the surface is moved and can stretch and retract. One embodiment of a film is elastomeric polyurethane, polyester, or polyether block amide films. These films combine the desirable properties of resiliency, high moisture vapor permeability, and transparency. Example of material for the thin film dressing is in 3M Tegaderm IV Dressings available from 3M Company. U.S. Patent application 62/783,368, filed Dec. 21, 2018 describes a medical article with backing that may be suitable as the contact dressing.

The medical dressing may be formed of or additionally include a nonwoven fibrous web, fibrous film web, knit, and other familiar backing materials. Additional reinforcing materials can be included with the thin film to give the dressing strength, rigidity. Examples of suitable nonwoven fabrics for use in the nonwoven soft-cloth reinforcement material included carded, spun-bonded, spun-laced, air-laid, blown microfibrous constructions, and stitch-bonded fabrics. Suitable commercially available substrate materials include kraft paper (available from Monadnock Paper, Inc.); cellophane (available from Flexel Corp.); spun-bond poly(ethylene) and polypropylene, such as TYVEK and TYPAR (available from DuPont, Inc.); and porous films obtained from polyethylene and poly(propylene), such as TESLIN (available from PPG Industries, Inc.), and CELLGUARD (available from Hoechst-Celanese).

Further examples of polymers which are suitable for use in the dressing body include polyurethane, elastomeric polyester such as DuPont “Hytrel” polyester elastomer (Wilmington, Del.), polyethylene, blends of polyurethane and polyester, chlorinated polyethylene, styrene/butadiene block co-polymers such as “Kraton” brand thermoplastic rubber (Shell Chemical Company, Houston Tex.), and polyvinyl chloride.

Absorbent Material

The medical dressing 100 can include an absorbent at the bottom major surface to absorb moisture or exudate. Common absorbent materials include woven, knitted, nonwoven fabrics, absorbent gels, superabsorbent materials, hydrocolloids. In some embodiments, the absorbent material contains an antimicrobial agent to reduce the likelihood of an infection. An example of an absorbent material containing an antimicrobial agent is the gel pad in the 3M Tegaderm CHG IV dressing, available from 3M Company, St. Paul Minn.

Adhesive

In one or more embodiments, adhesives used for the various components of the dressing 100 may be skin friendly adhesives useful in securing a medical dressing to the skin of a patient. In one or more embodiments, the adhesive s 138 (label), 134 a, b (fixation strips), 153 (film layer) and 142 (reinforcing material) used in the dressing 100 may be the same adhesives. In one or more alternative embodiments, the adhesives used throughout the construction of the dressing 100 may be different. Although the adhesives are depicted as solid, continuous layers, it will be understood that adhesives used in connection with the medical dressings described herein may be discontinuous (e.g., pattern coated) as is commonly known.

Suitable adhesives for use in one or more embodiments of the medical dressings described herein include any adhesive that provides acceptable adhesion to skin and is acceptable for use on skin (e g., the adhesive should preferably be non-irritating and non-sensitizing). Suitable adhesives are pressure sensitive and in certain embodiments have a relatively high moisture vapor transmission rate to allow for moisture evaporation. Suitable pressure sensitive adhesives include those based on acrylates, urethane, hydrogels, hydrocolloids, block copolymers, silicones, rubber-based adhesives (including natural rubber, polyisoprene, polyisobutylene, butyl rubber, etc.) as well as combinations of these adhesives. The adhesive component may contain tackifiers, plasticizers, rheology modifiers as well as active components including, for example, an antimicrobial agent.

The pressure sensitive adhesives that may be used in the dressings described herein may include adhesives that are typically applied to the skin such as the acrylate copolymers described in U.S. Pat. No. RE 24,906. particularly a 97:3 isooctyl acrylate:acrylamide copolymer. Another example may include a 70:15:15 isooctyl acrylate:ethyleneoxide acrylate:acrylic acid terpolymer, as described in U.S. Pat. No. 4,737,410 (Example 31). Other potentially useful adhesives are described in U.S. Pat. Nos. 3,389,027; 4,112,213: 4,310,509; and 4,323,557. Inclusion of medicaments or antimicrobial agents in the adhesive is also contemplated, as described in U.S. Pat. Nos. 4,310,509 and 4,323,537.

Suitable silicone adhesive can also be used. Generally, silicone adhesives can provide sufficient adhesion to skin while gently removing from skin. Suitable silicone adhesives are disclosed in PCT Publications WO2010/056541 and WO2010/036543.

The pressure sensitive adhesives may, in some embodiments, transmit moisture vapor at a rate greater to or equal to the transepidermal water loss of the human skin. While such a characteristic can be achieved through the selection of an appropriate adhesive, it is also contemplated that other methods of achieving a high relative rate of moisture vapor transmission may be used, such as pattern coating the adhesive, as described in U.S. Pat. No. 4,395,001. The pressure sensitive adhesive used in the dressing may also include one or more areas in which the adhesive itself includes structures such as, e.g., the microreplicated structures described in U.S. Pat. No. 6,893,655. U.S. Pat. Nos. 3,645,835 and 4,595,001 describe methods of making such films and methods for testing their permeability. Different portions of the dressings 100 described herein may include different adhesives, such as disclosed in U.S. 2015/0141949 titled “Medical Dressings with Multiple Adhesives”. For example, a portion may include an acrylate adhesive and another portion may include a silicone adhesive.

Release Liner

Suitable release liners can be made of paper or film such, as kraft papers, polyethylene, polypropylene, polyester or composites of any of these materials. The release liner is typically coated with release agents such as fluorochemicals or silicones. The release coating can be on one side or on both sides. In an embodiment, the release agent is applied to the line only in areas which will contact the substrate to be protected. For example, U.S. Pat. No. 4,472,480, the disclosure of which is hereby incorporated by reference, describes low surface energy perfluorochemical liners. In one embodiment, the liners are papers, polyolefin films, or polyester films coated with silicone release materials. Additionally, the release liner may be printed one or both sides. Examples of silicone coated release papers are Polysilk™ silicone release papers supplied by Daubert Chemical Co., Dixon Ill.

Carrier

Suitable materials utilized for the carrier are similar to the materials described above for the liner, although typically a thicker cut of material is used. A release coating is also utilized to help the carrier material release from the film of the dressing body and is applied to the surface in contact with the dressing body. In an embodiment, a two-sided carrier is utilized to further ensure ease of separation from packaging materials.

Fixation Strips

The fixation strips may be made of any material that can be coated with adhesive and function as a securement device. In an embodiment, a nonwoven material is used with an adhesive coated on one side of the material. For example, tapes such as 3M Micropore™ tape, 3M Medipore™ a tape, 3M 3M Transpore™ a tape could be used as the fixation strip.

Label

Various medical tapes may be suitable for use as a label material. The label may have printed information to aid the user in entering patient information. The label may be formed of a material that can be written on with a pen or marker. In an embodiment, 3M Micropore™ tape or 3M Medipore™ tape can be used as a label material.

Although specific embodiments have been shown and described herein, it is understood that these embodiments are merely illustrative of the many possible specific arrangements that can be devised in application of the principles of the invention. Numerous and varied other arrangements can be devised in accordance with these principles by those of skill in the art without departing from the spirit and scope of the invention. The scope of the present invention should not be limited to the structures described in this application, but only by the structures described by the language of the claims and the equivalents of those structures.

1. A medical dressing comprising:
a dressing body having a top major surface and a bottom major surface opposite the top major surface, and a dressing perimeter defining a dressing area, wherein the dressing area has a first dressing area and a second dressing area;
a pressure sensitive adhesive on at least a portion of the bottom major surface of the dressing body;
a carrier releasably secured to the top major surface of the dressing body, wherein the carrier comprises a central carrier portion and a peripheral carrier portion;
wherein the peripheral carrier portion is adjacent to the perimeter of the dressing body;
wherein the central carrier portion is adjacent to the first dressing area;
wherein the second dressing area is free of the carrier.

2. The medical dressing of claim 1, wherein the second dressing area is at least partially surrounded by the peripheral carrier portion and the central carrier portion.

3. The medical dressing of claim 1, wherein at the second dressing area, the dressing body forms an outermost surface of the medical dressing.

4. The medical dressing of claim 1, wherein the dressing body further comprises a slot, which is a recess of the dressing perimeter at the second dressing area.

5. The medical dressing of claim 1, wherein the dressing body is a single or multilayer film.

6. The medical dressing of claim 5, wherein the multilayer film of the dressing body at the first dressing area comprises a moisture-vapor permeable film and at the second dressing area comprises a reinforcing layer and a moisture vapor permeable and water impermeable film.

7. The medical dressing of claim 1, wherein the pressure sensitive adhesive is on the entire bottom major surface of the dressing body.

8. The medical dressing of claim 1, wherein the pressure sensitive adhesive is in a discontinuous pattern on the bottom major surface of the dressing body.

9. The medical dressing of claim 1, further comprising an absorbent on the bottom major surface.

10. The medical dressing of claim 1, wherein at least a portion of the central carrier portion is directly adjacent to the peripheral carrier portion.

11. The medical dressing of claim 10, wherein the central carrier portion is connected with the peripheral carrier portion by an area of weakness.

12. The medical dressing of claim 11, wherein the area of weakness is a partial thickness cut or a perforation.

13. The medical dressing of claim 1, wherein the central carrier portion further comprises a central carrier tab extending into the second dressing area.

14. The medical dressing of claim 1, wherein the peripheral carrier portion further comprises a peripheral carrier tab extending into the second dressing area.

15. The medical dressing of claim 1, wherein the peripheral carrier portion extends to the dressing perimeter.

16. The medical dressing of claim 1, further comprising an adhesive fixation strip at the top major surface secured to the central carrier portion and the peripheral carrier portion.

17. A method applying the medial dressing of claim 1, comprising the steps of:
placing the pressure sensitive adhesive of the dressing body onto a surface;
removing the central carrier portion; and
removing the peripheral carrier portion.

18. A method applying the medial dressing of claim 1, comprising the steps of:
removing the central carrier portion;
placing the pressure sensitive adhesive of the dressing body onto a surface;
removing the peripheral carrier portion.

19. The method of claim 17, further comprising pulling the central carrier tab to remove the carrier central portion.

20. The method of claim 17, further comprising pulling the peripheral carrier tab to remove the peripheral carrier portion.
